(19) United States
(12) Patent Application Publication
(10) Pub. No.: US 2014/0121627 A1
(43) Pub. Date: May 1, 2014

(54) COMPRESSIVE DRESSING AND PRODUCTION PROCESS THEREOF

(71) Applicants: Emiliano Lepore, Torino (IT); Roberto Capocelli, San Mauro Torinese (TO) (IT)

(72) Inventors: Emiliano Lepore, Torino (IT); Roberto Capocelli, San Mauro Torinese (TO) (IT)

(73) Assignee: Emiliano Lepore, Torino (IT)

(21) Appl. No.: 14/124,782

(22) PCT Filed: May 29, 2012
(86) PCT No.: PCT/IB2012/052666

§ 371 Date: Dec. 20, 2013

(30) Foreign Application Priority Data
Jun. 8, 2011 (IT) TO2011A000499

(51) Int. Cl. A61F 13/02

(52) U.S. Cl. CPC A61F 13/0273; A61F 13/0206; A61F 13/0289
USPC 604/385.01; 493/340

(57) ABSTRACT

Compressive dressing (1) applicable in a single winding to a body part, the dressing (1) having an inner surface (70) and an outer surface (71) essentially having the same area; the dressing (1) comprising:—A multilayer structure comprising: i) an inner elastic layer (10) defining said inner surface (70) and provided for contact with the skin, the inner layer (10) being hydrophilic, non-adhesive to the skin and of absorbing material, ii) an elastic intermediate layer (20) permeable to gases and impermeable to liquids, and iii) a flexible outer layer (30) defining the said outer surface (71), wherein the multilayer structure has at least a first edge region (40) and a second edge region (50) generally opposite one other, and—closing formations (60) which can be mutually coupled at the first edge region (40) and the second edge region (50) to close the multilayer structure in a single winding on the body part of the body.

### FIELD OF THE INVENTION

The present description relates to a compressive dressing and a relative production process.

### BACKGROUND OF THE INVENTION

Wound dressings are used to protect the damaged area from possible environmental contamination, as well as to promote and maintain the conditions necessary to ensure that the complex process of tissue repair can begin and continue unhindered until the healing of the lesion.

For obtaining these objectives, dressings may be formed from several layers with distinct properties.

In this context, the patent documentation is extensive. For example, patent documents EP-A-0437994, EP-A-0878179, EP-A-1709947, U.S. Pat. No. 4,360,015, U.S. Pat. No. 4,561,435, U.S. Pat. No. 5,939,339, US-A-2001/0029346, US-A-2005/0182347, US-A-2007/0179522, US-A-2007/0185428, US-A-2009/0112141, WO-A-84/03832, WO-A-02/15816, FR-A-2662361 describe multilayer wound dressings for covering and protecting lesions of various kinds that can be characterized by the release of exudates.

Despite extensive research as documented at a patent level, only a small number of operative solutions have a practical and effective use. This fact is due to the difficulty of making dressings that simultaneously satisfy all the following requirements:
maintaining a constant and correct gradient in the moist bed of the lesion;
allowing the gaseous exchange of oxygen, carbon dioxide and water vapour with the surrounding environment;
possessing a high absorbent capacity over the whole extension of the dressing with respect to exudates released from the wound;
maintaining a constant physiological temperature in the wound bed;
should be impermeable to the entrance of microorganisms, liquid and dirt from the outside;
should not adhere to the damaged skin;
guaranteeing a mechanical protection of the injured area;
should be comfortable, conformable and flexible;
should be manageable without causing pain during application and removal.

The solutions described in the patent documents mentioned above however, only satisfy some of the features listed above and in some cases have additional disadvantages.

For example, in some cases (US-A-2009/0112141, WO-A-02/15816, U.S. Pat. No. 5,939,339) the absorbent material is only located in a portion of the dressing, for example in the vicinity of one end of the dressing, and this aspect makes the dressing not very versatile with respect to lesions that extend over a large body surface area.

The dressings can be made of materials such as gauze (FR-A-2662361), but such material is not always able to effectively dissipate moisture and is easily saturable, becoming a favourable site for the development of bacteria, and thus requiring frequent removal.

Some dressings include the use of adhesive materials in contact with the lesioned (EP-A-0437944) or perilesional (U.S. Pat. No. 4,561,435) skin. However, adherence to the wound can result in pain during dressing removal and can cause damage to newly formed tissue with a slowing of the healing process. In addition, adhesive bandages may be inconvenient since they are generally equipped with a low elasticity; this causes the adhesive layer to exert a traction force on the lesioned skin during movement, damaging the skin and causing pain.

Some dressings are made to be wrapped around a body portion and adhere onto themselves (WO-A-02/15816). These bandages may present the disadvantage of not adhering adequately to the portion to be treated, and therefore, as a result of mechanical stresses due to movement, undergo displacement along the injured body surface.

Other dressings require devices such as adhesives or hooks in order to be fixed (U.S. Pat. No. 4,360,015, US-A-2007/0179522, US-A-2007/0185428) or are worn by pulling them on, for example on a limb (WO-A-84/03832). This solution can be particularly disadvantageous in that the sliding on of a dressing on an injured body region can cause pain, irritation and further damage to already damaged skin.

Furthermore, in some cases dressings do not exert any compression (US-A-2005/0182347) and may require a secondary dressing (US-A-2001/0029346) placed on top of the other dressing for this purpose.

In other cases, dressings are capable of exerting compression, but for this purpose they must be applied by multiple winding on the body portion to be dressed (WO-A-02/15816, EP-A-1709947 and EP-A-0878179). Such dressings may include separate layers, a first layer in contact with the skin of absorbent material and a second outer elastic layer. These dressings are wrapped around the body portion to be treated in two stages: first the inner absorbent layer is wrapped around followed by the outer layer. The windings are spiral and the level of compression in this type of dressings is modulated by the windings.

Dressings of this type have several drawbacks. Firstly, the winding of the dressing too tightly around the anatomical portion to be treated compresses and exerts excessive, poorly distributed and damaging pressure. In addition, the multiple winding is difficult to apply and may present windings with different compression capacity between them and thus rendering it unable to perform its function.

In addition, these dressings are particularly costly especially in hospital settings, as they require the intervention of numerous human resources simultaneously engaged in the dressing phase of the patient. In fact, very often the patient alone is not able to perform all the operations necessary to correctly and effectively wind the dressing around the body region body to be treated.

To position the dressing properly, therefore, requires the intervention of qualified personnel as a an incorrect procedure, such as non-close-fitting positioning causes displacement of the dressing especially when the area of the body to be dressed is subjected to mechanical stress, such as for example a joint.

### SUMMARY OF THE INVENTION

Taking into account this background, the need for improving solutions is therefore felt, the most effective being those that allow provision of a compressive dressing capable of absorbing exudates released by a lesion, favouring the passage of gases and preventing the passage of liquids, easy to apply and to remove and able to exert a uniform compression over the entire area to be treated.

In accordance with the invention, the said object is achieved thanks to the solution specifically outlined in the accompanying claims, which constitute an integral part of the present description.

One embodiment of the present invention relates to a compressive dressing applicable in a single winding on a body part, the dressing having an inner surface and an outer surface with essentially the same area and including:
A multilayer structure comprising:

i) an elastic inner layer defining said inner surface, and provided for contact with the skin, the elastic inner layer being hydrophilic and of absorbent material,

ii) an elastic intermediate layer, permeable to gases and impermeable to liquids, and

iii) a flexible outer layer, defining said outer surface,

wherein the multilayer structure has a first edge region and a second edge region generally opposite one other, and
closing formations which can be mutually coupled at said first and second edge region to close the multilayer structure as a single winding on said body part.

A second embodiment of the present invention relates to a production process, preferably but not necessarily, of the compressive dressing described above with anatomical conformation. The method of embodiment comprises:

i) provision of a piece of a multilayer structure having at least four edge regions of the edge, wherein a first edge region is generally opposite to a second edge region, and a third edge region is generally opposite to a fourth edge region;

ii) implementing at least one intermediate slit in the piece by removal of a respective excess portion of the piece, in such a way whereby at least two flaps are defined in the piece, generally side by side and separated by said slit, wherein said at least one slit is created by making at least two intermediate cutting lines in the piece which extend from the fourth edge region of the piece towards the third edge region of the piece,

iii) approximating said at least two flaps to one another and binding them to each other along the two cutting lines of the respective slit in the absence of a substantial overlap between the flaps themselves, or with the two cutting lines essentially facing each another, resulting in a compressive dressing with an anatomical conformation.

### BRIEF DESCRIPTION OF THE DRAWINGS

The invention will now be described in detail, purely by way of an illustrative and non-limiting example and, with reference to the accompanying drawings, wherein:

FIG. 1 is a perspective view of one embodiment of the compressive dressing subject of this description;

FIG. 2 is a different perspective view of one embodiment of the compressive dressing subject of this description;

FIGS. 3a and 3b illustrate how to apply a compression dressing according to the present description on a forearm;

FIGS. 4 to 6 depict a mode of production of one embodiment of the compression dressing 1 in an anatomical conformation for the application on a calf;

FIG. 7 shows the application of a multiplicity of compression dressings over the entire body surface of a subject;

FIG. 8 schematically shows three different application conditions of the dressing, subject of the present invention, on a body part to be dressed, wherein the dressing is in an essentially unstressed condition.

### DETAILED DESCRIPTION OF THE INVENTION

In the following description, numerous specific details are presented to provide a complete understanding of the embodiments. The embodiments may be implemented in practice without one or more of the specific details, or by other processes, components, materials, etc. In other cases, well known structures, materials, or operations are not shown or described in detail to avoid obscuring certain aspects of the embodiments.

Throughout the present specification, the reference to “one embodiment” or “embodiment” means that a particular feature, structure, or characteristic described in connection with the embodiment is included in at least one embodiment. Thus, the occurrence of the expressions “in a certain embodiment” or “in one embodiment” in various sites throughout the present specification does not necessarily always refer to the same embodiment. Furthermore, the particular features, structures, or characteristics may be combined in any suitable manner in one or more embodiments.

The titles used herein are merely for convenience and do not interpret the object or the meaning of the embodiments.

Where not otherwise specified, in the description which follows and in the accompanying claims, the expression “in a single winding” and similar expressions, essentially means the presence of a single winding or entwinement of the dressing on the body portion to be dressed, or the absence of overlap of two or more entwinements of the dressing on itself.

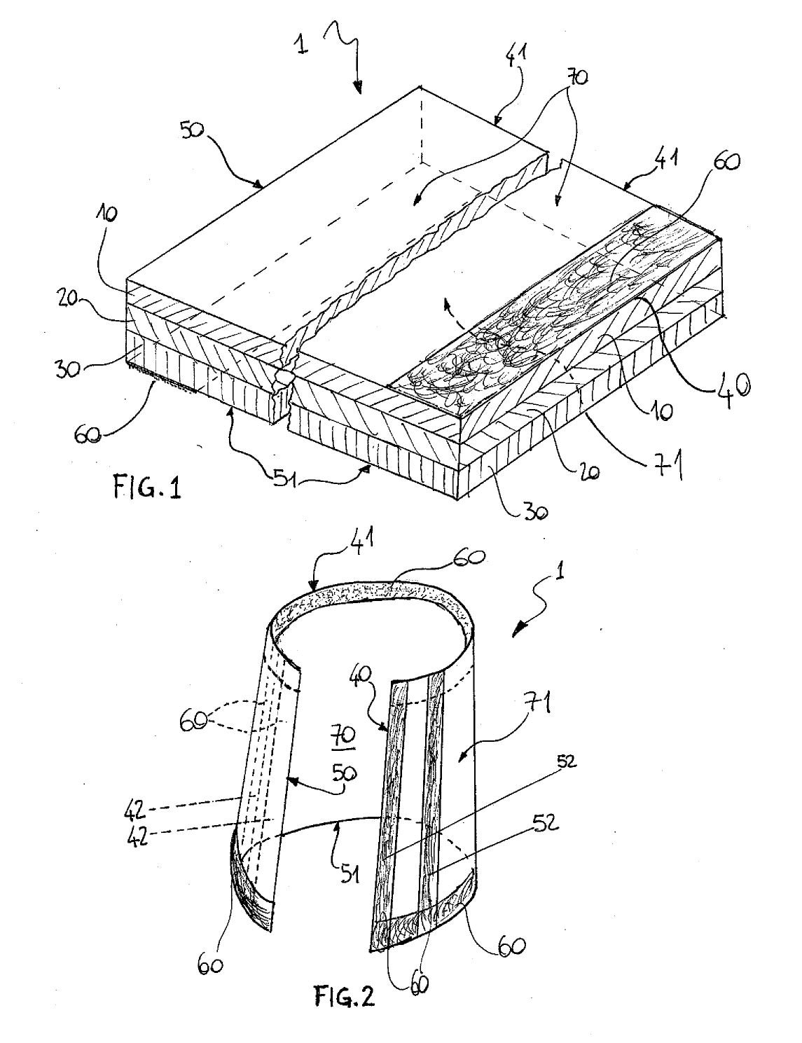

One embodiment of a compressive dressing according to the present invention is schematically illustrated in FIG. 1. Note that in this figure the dimensioning of the various parts, and in particular their thickness, is greatly enlarged for increased clarity of representation.

The compressive dressing, indicated as a whole with 1, has an inner surface 70 and an outer surface 71, essentially having the same area. The dressing is applicable in a single winding on a part of the human or animal body, so that the inner surface 70 is in contact with the skin. The dressing 1 comprises:
a multilayer structure that comprises i) an elastic inner layer 10—defining the surface 70 intended for contact with the skin—made of hydrophilic, non-adhesive and absorbent material, ii) an elastic intermediate layer 20 permeable to gases and impermeable to liquids and iii) a flexible outer layer 30, defining the outer surface 71 of the dressing. The multilayer structure has at least two edge regions generally opposite each other, including a first 40 and a second edge region 50, and closing formations 60, mutually coupled at said first edge region 40 and second edge region 50, to close the multilayer structure in a single winding on said body part. The distance between the first and the second edge region 40 and 50 corresponds to the length l of the dressing 1, this length being associated with a transverse direction.

The multilayer structure also presents at least two edge regions generally opposite each other, including a third and a fourth edge region 41 and 51. The distance between the third and the fourth edge region 41 and 51 corresponds to the height h of the dressing 1, this height being associated with a longitudinal direction.

The compressive dressing 1 is suitable for dressing of skin lesions of various kinds, and in particular of skin lesions that release exudates.

As said, dressing 1 has an inner surface 70 applicable to the skin of the body portion to be dressed and an outer surface 71, opposite to the inner surface, intended to protect the body portion from any external insults. Note that the entire inner surface 70 is defined by the elastic inner layer 10, of which hydrophilic material is intended to cover both the lesion to be repaired and the area of skin that surrounds it. Even more particular, the inner surface 70, i.e. the layer 10, is intended for winding round the section of the limb or trunk in which the lesion is localized.

The compressive dressing 1 presents values of absorbent capacity (i.e. hydrophilicity) in the range of 0.1 l/m² to 5 l/m², preferably 0.5 to 2 l/m².

The compressive dressing 1 presents Resistance of Evaporation of a Textile (RET) values in the range of 0.1 to 100 m²·Pa/W, preferably less than 10 m²·Pa/W (according to standard UNI EN 31092:1996). The dressing 1 also presents values of resistance to the passage of water no less than 1 cm H₂O, preferably greater than 300 cm H₂O, preferably in the range 10-5000 cm H₂O, (according to standard UNI EN 20811:1993).

The compressive dressing 1 is able to exert a compression of the order of 1-60 mm Hg on the body portion wrapped by it.

The hydrophilic elastic inner layer 10 is made of absorbent material, ensuring optimal drainage of exudates from the wound, protecting both the damaged skin and skin adjacent to the lesion, and—in general—the section of the trunk or the limb presenting the lesion.

The ability of the elastic inner layer 10 not to adhere to the skin allows removal of the compressive dressing 1 without causing damage to the skin itself during its removal, i.e. without damaging the generated scar tissue around and/or on the lesion.

To enhance the absorptive function of the fluid which escapes from the lesion such a layer can in turn be constituted by two or more layers of elastic hydrophilic absorbent material.

The elastic intermediate layer 20 is permeable to gases, such as water vapour, carbon dioxide and oxygen, and impermeable to liquids.

The correct levels of permeability to gases and impermeability to liquids represent important features of the compressive dressing 1, subject of the present description, since they allow the generation of a moist and warm environment at the level of the lesion with a degree of humidity and temperature sufficient to facilitate the healing process. The microenvironment that is generated on the lesion and in the area adjacent to it in fact promotes the generation of granulation tissue on the wound bed, or rather of tissue originated by cell proliferation of the epithelial structures, endothelial cells and connective tissue during the healing process of the skin lesion.

The elastic intermediate layer 20, by maintaining the level of gas exchange at physiological values, prevents, on the one hand, an excess of water vapour release, which would lead to dehydration of the tissue wrapped by the dressing 1 with consequent loss of elasticity, and on the other prevents an excessive accumulation of gases (in particular water vapour and carbon dioxide) which would lead to a process of maceration and necrosis of the tissue wrapped by the dressing 1. In addition, the microenvironment generated—allowing the exchange of oxygen with the outside of the dressing—favours the autolysis of the necrotic tissue, which is the process by which endogenous proteolytic enzymes are able to liquefy and separate the necrotic tissue from healthy tissue in a physiological manner.

Again, the elastic intermediate layer 20, being impermeable to liquids, allows containment of exudates from the lesion in the inner part of the dressing thus preventing their escape to the outside, with consequent need for frequent replacement of the dressing.

These properties of permeability to gases and impermeability to liquids are achieved thanks to the presence of pores in the elastic intermediate layer 20 which have dimensions suitable for the passage of gaseous molecules and for the retention of liquid exudates from the wound. In particular, the pores present in the elastic intermediate layer 20 have a size in the range of 1 nm-10 μm, preferably 0.01 to 1 μm.

The flexible outer layer 30 allows application of a homogeneous compression—in cooperation with the elastic inner layer 10 and intermediate layer 20—uniformly on the body portion wrapped in the compressive dressing 1, so as to permit proper healing of the lesion. This layer does not, however, constitute a barrier to gas exchange.

In a preferred embodiment of the compressive dressing 1, between the elastic inner layer 10 and the elastic intermediate layer 20, or between the elastic intermediate layer 20 and the flexible outer layer 30, further layers may be present, equipped with absorbent capacity.

The elastic inner layer 10, intermediate layer 20 and outer layer 30 each have a respective uniformity of thickness, elasticity and compressive action.

As said, the compressive dressing 1, subject of the present description is made to be wrapped around a body part and is fixed thereto by means of closing formations 60 which can be mutually coupled at two edge regions 40 and 50 of the multilayer structure, opposite to each other.

In one preferred embodiment, the closing formations 60 can be made with Velcro, where at least a first strip of fabric 42, said loop, is applied to the surface 70—i.e. at the elastic inner layer 10—at the first edge 40, and at least a second strip of fabric 52, said hook, is applied to the surface 71, not in contact with the skin—at the flexible outer layer 30—at the second edge 50. The fabric strips 42 and 52 may of course be applied on the multilayer structure in a contrary manner with respect to what is described above.

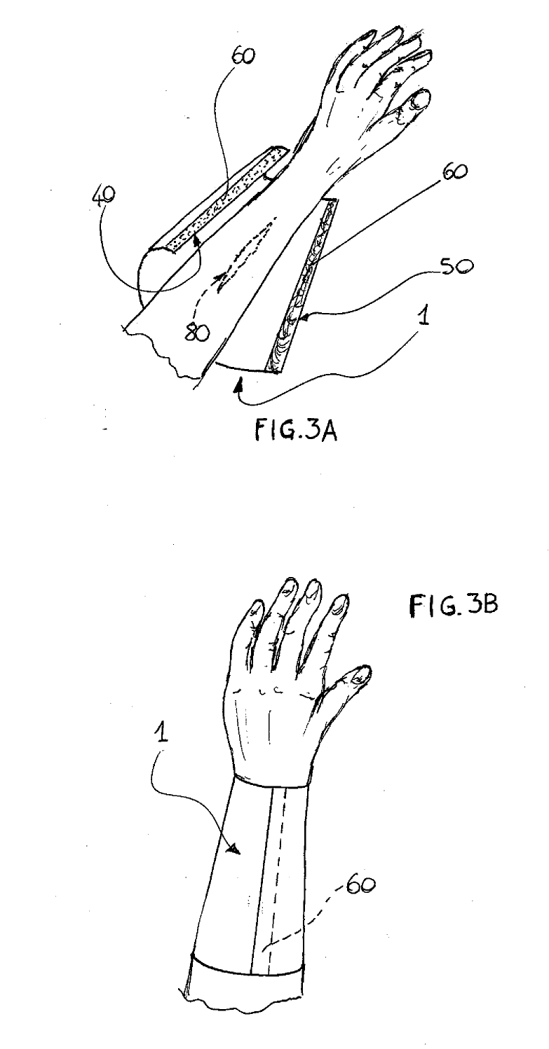

The closing formations 60 can be applied in single, double (as shown schematically in FIG. 2) or multiple strips, along the entire extension of the edges 40, 50 of the multilayer structure or along their fractions.

Further closing formations 60 may also be applied on a third edge region 41 and a fourth edge region 51 generally opposite to each other in the multilayer structure, at the inner surface 70 and the outer surface 71, respectively; preferably, therefore, said further closing formations 60 extending in a direction generally transverse with respect to the closing formations provided on the first and second edge 40, 50. The presence of such additional closing formations 60 on the third and fourth edge of the multilayer structure is useful in the case wherein it is necessary to employ more than one compressive dressing 1 for winding around the body portion to be dressed. In other words, by exploiting the presence of closing formations which can also be mutually coupled at edges other than those indicated with 40 and 50, it is possible to “arrange” between them more dressings 1, in order to wrap—for example—an entire limb and not only a section, with no interruption of protection between one dressing and the next.

The use of closing formations 60 that can be mutually coupled for the closure of the dressing 1 in a single winding on the body part to be dressed allows easy opening of the dressing and at the same time, when necessary, a solid and resistant closure.

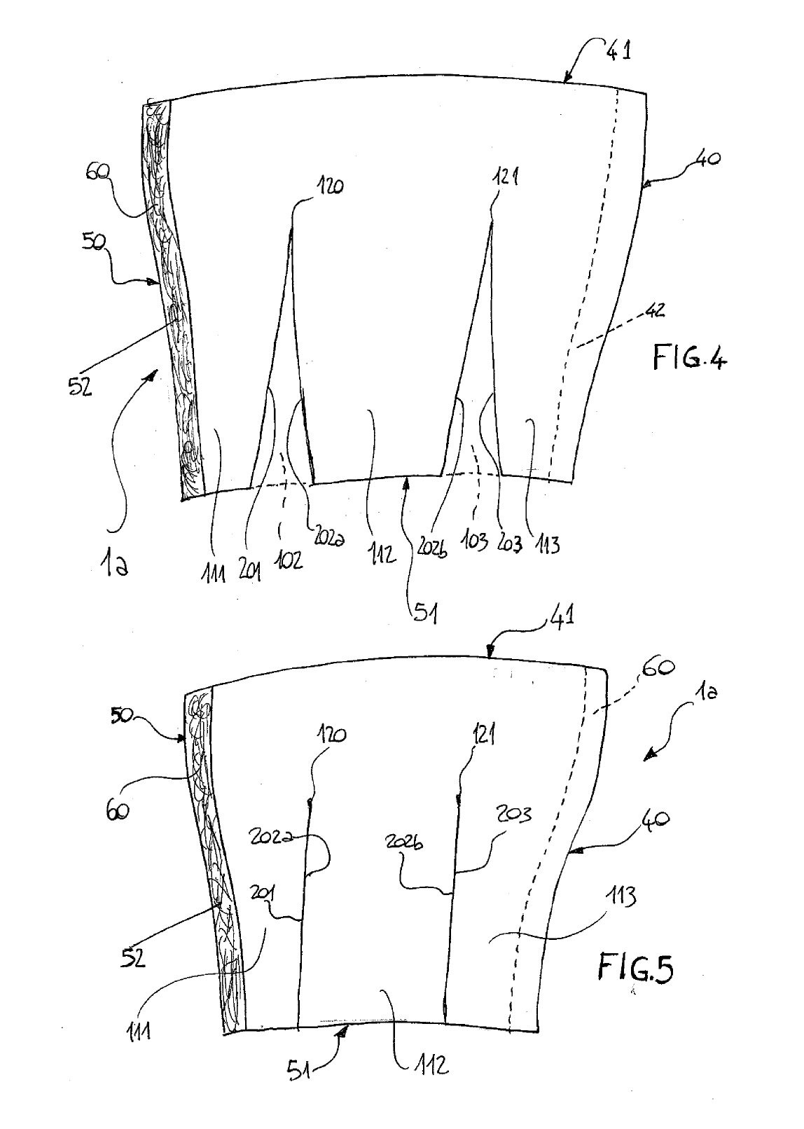

As shown in FIG. 3a, in order to be worn, dressing 1 is affixed to cover the portion of the body to be treated which has a lesion 80 and is then closed by coupling the closing formations 60, as seen in FIG. 3b; note that in FIG. 3a, the lesion 80 is represented by dashed lines to indicate that it is located on the limb depicted from the side opposite to that in the foreground. In this way, avoiding having to wear the dressing by “inserting” it onto the part of the body to be dressed, which prevents further damage to the lesioned skin which could cause painful chafing.

Thanks to the closing formations of 60 which can be mutually coupled, it is possible to wear and close dressing 1 with ease guaranteeing a perfect adherence of the dressing and the application of a homogeneous compression to the body segment to be treated, as shown in FIG. 3b.

The dressing 1, moreover, is able to ensure protection of the lesion by mechanical insults such as pressure, shock, friction and acts as an impermeable barrier against liquid and external dirt.

At the time of removal/replacement of the dressing, the patient or he/she who has to remove/replace the compressive dressing 1 will exert a force required to separate the closing formations of 60. In this way, the removal of the dressing will take place in an untraumatic and painless manner, without damage to the newly formed tissue and without leaving residues on the lesion thanks to its ability not to adhere to the skin of the elastic inner layer 10.

The dressing 1 of the present description is able to exert a homogeneous and uniform compression (in the order of 1-60 mm Hg) on the body portion wrapped by the dressing 1.

The compression in the range of 1-60 mm Hg on the body part intended to be wrapped by the compressive dressing 1 is made by selecting the distance between the first and the second edge region 40 and 50 in a suitable way (i.e. the length l of the dressing 1 itself) and/or the distance between the closing formations 60 and/or the overall elasticity ΔL % of the multilayer structure. Naturally, for the same portion of the body—for example, a calf—various sizes of the dressing 1 can be prepared, in order to have dressings suitable for patients with different physical structures (e.g. a small size, a medium size and a large size).

This homogeneity and uniformity of compression is obtained thanks to the essential absence of superimposed portions, which is extended over a significant area of the multilayer structure when it is applied in a single winding on the body portion to be dressed. An overlap is only provided at the closing formations 60.

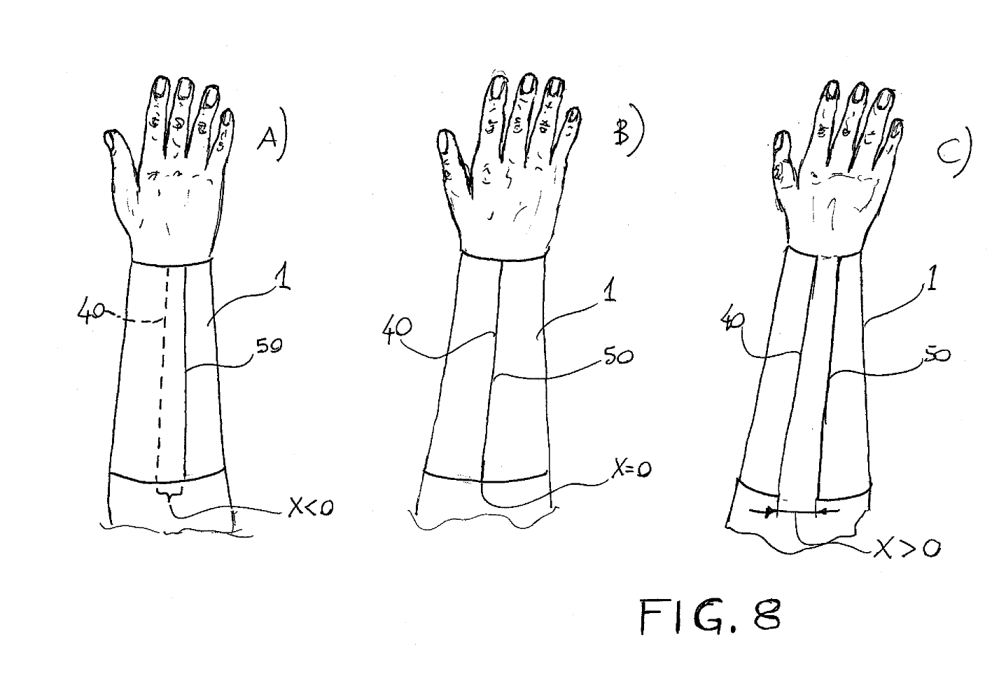

With reference to FIG. 8, in order to better understand the possibilities offered by dressing 1 in terms of choice of the magnitude of the compression action, the distance is introduced between the edge regions 40, 50 of the dressing 1 when applied in an essentially unstressed condition on the body part to be dressed, indicating it with X. By this it means that dressing 1 has been applied so as to essentially conform to the geometry of said part of the body without imposing the same further deformations to the dressing—applying and/or generating stresses on the dressing—beyond those necessary for the simple adaptation to the geometry of the body part to be dressed. In other words, it is assumed that dressing 1 is simply “placed” on the body part and is adapted to the geometry of it before closing.

In such cases it is possible to identify three fields of characteristic values for the distance X, i.e.:
1. X<0: dressing 1 has dimensions (length l) such as to essentially encircle the body part on which it is applied with an overlap of the edge regions 40, 50 in an unstressed condition (FIG. 8a);
2. X≈0: dressing 1 has dimensions (length l) whereby it is able to surround the body part on which it is applied with a substantial contact “head-to-head” of the edge regions 40, 50, essentially without overlapping them (FIG. 8b);
3. X>0: dressing 1 has dimensions (length l) such that it cannot completely encircle, in unstressed conditions, the body part to be dressed and there is no contact between the edge regions 40, 50 (FIG. 8c).

The size of the dressing, in particular the length l, can then be chosen so that the distance X results in having values belonging to one of the three above-mentioned fields, which offers the possibility to apply compression actions of different entities. In particular, the magnitude of the compression action may have values belonging to the following intervals:
a. approximately between 1 and 10 mm Hg in the case of distances X=X₁<0 or X=X₁≈0;
b. approximately between 10 and 20 mm Hg in the case of distances X=X₂≈0 or X=X₂>0;
c. approximately between 20 and 30 mm Hg in the case of distances X=X₃>X₂>0;
d. approximately between 30 and 60 mm Hg in the case of distances X=X₄>X₃>0.

In each case it is possible, given the starting size—that is, length l—of the dressing 1, to modulate the magnitude of the compression action in the above-mentioned ranges operating on:
elasticity (ΔL %) of the dressing; and/or
number of closing formations 60.

In general the size of the undeformed dressing 1 is chosen knowing already the magnitude of the compression action that will be applied to the body part to be dressed. That is, an oversized dressing (X<0 or X≈0) is chosen if the objective is to apply a compression action of magnitudes in the range of 1 and 10 mm Hg.

It is not, however, convenient to choose an oversized dressing to apply high values of pressure (e.g. 20-60 mm Hg), in that the choice of the elasticity values of (Δl %) of the dressing 1 and/or the number of closing formations 60 could be extremely complex and, moreover, could give rise to an unwanted overlapping of a large area of the dressing 1. This essentially explains the association of the values of the distance X to the ranges of pressure values listed above.

Homogeneous and uniform compression exerted by the dressing 1 when applied to the body part to be dressed favours good healing of the lesion, limiting any bleeding, and promoting the reabsorption of fluid exudates from the wound.

Dressing 1 is also anatomically shaped, or rather has a three-dimensional profile suitable to be applied in a single winding on the body part to be dressed: in other words, even before being applied, dressing 1 has a profile congruent to the anatomical shape of the body part to be dressed. In this perspective, and as we shall see later, dressings 1 may for example be prepared for various parts of the body of a man or an animal, such as the face, hands, feet, knees, thighs, calves, forearms, abdomen, etc.

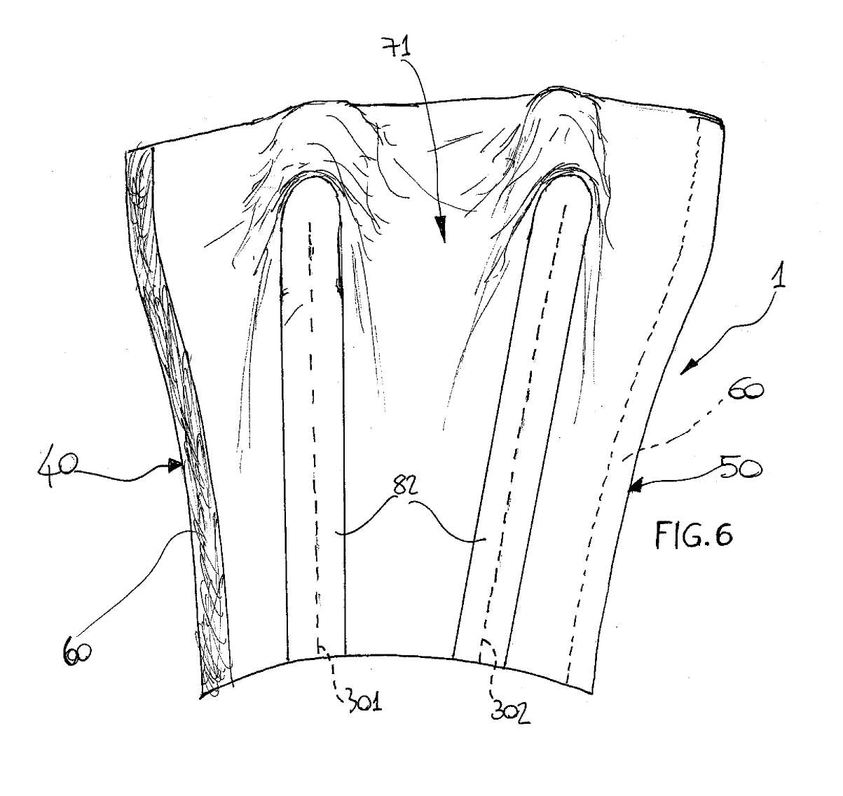

In FIGS. 4 to 6 one way of producing a compressive dressing 1, shaped for a calf of a man, is schematically shown, purely by way of an illustrative and non-limiting example.

As said, in fact, dressing 1 is achievable in different versions, each arranged to conform to a respective body part, such as an arm, a forearm, abdomen, thigh, knee, and so on. Dressing 1 is also conformable to irregular anatomical surfaces, thanks to the flexibility of the layers constituting the multilayer structure.

FIG. 4 shows a semi-finished product of the dressing, and in particular a piece of multilayer structure 1a; for simplicity, this piece is represented as already being equipped with the closing formations 60, taking for granted, however, that the same can be applied in a final step of the production process.

As can be seen, the multilayer piece 1a exhibits longitudinal cuts, which give rise to three flaps 111, 112 and 113. These flaps are progressively matched to each other along respective edges 201 and 202a, 202b and 203, as shown in FIG. 5, to give origin to a three-dimensional profile of the compressive dressing 1, shown in FIG. 6, able to be wrapped around a calf following the anatomical conformation.

The flaps 111, 112 and 113 are joined together essentially without overlapping the respective edges 201 and 202a, 202b and 203. The resulting bonding zones 301 and 302 of the flaps 111, 112, 113 are, in one preferred embodiment, then covered, at the flexible outer layer 30 (i.e. at the surface 71 not in contact with the skin), with a bonding tape 82 along their entire length as shown in FIG. 6. The tapes 82 may also be applied in a continuity solution along the edges 201 and 202a, 202b and 203 of the flaps 111, 112 and 113 (i.e. with more strips spaced apart along the development of these said edges pulled together).

Dressing 1 is made so as to minimize, and preferably avoid, areas of overlap between the flaps of the multilayer structure, i.e. at the edges 201 and 202a, 202b and 203 of the flaps 111, 112 and 113.

The compressive dressing 1 is preferably carried out without the use of stitching.

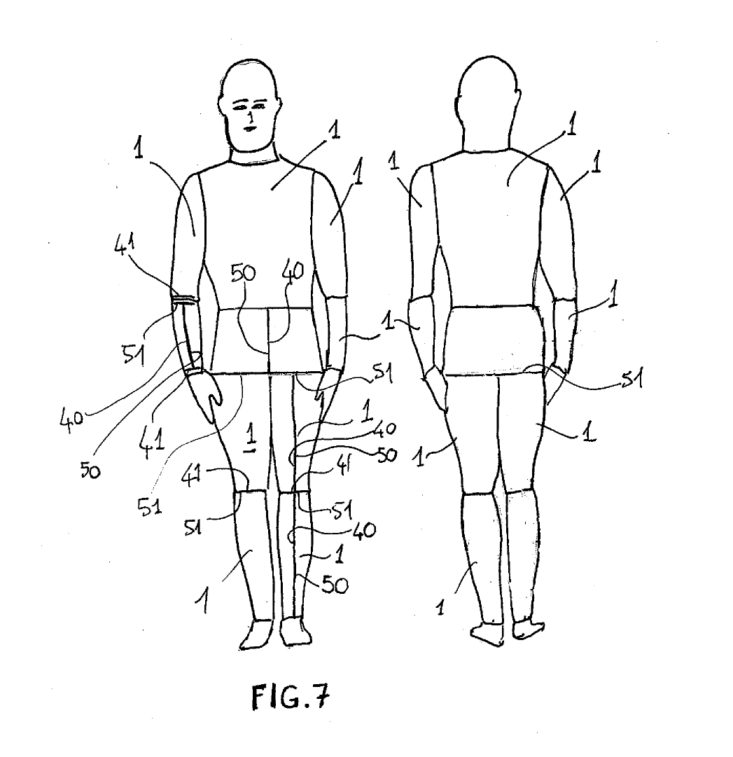

The compressive dressing 1 is comparable to a second skin, and therefore may consist of a total segmented coverage of each body area. In this case—as already mentioned—a dressing 1 can be produced to be interconnected with one or more additional dressings 1. For example, the closing formations 60 can be positioned along the third 41 and the fourth edge 51, opposite to each other, as shown in FIG. 2, so that two dressings 1, similar to that represented may be coupled together in the axial direction: proceeding in this way it is possible to produce a homogeneous, uniform, conformable, anatomical, even almost total coverage of the body, as shown in FIG. 7.

The compressive dressing 1 subject of the present description is used, by way of a non-limiting example, for the treatment of lesions (also extended) with or without eventual loss of substances, mild exudatives (e.g., skin abrasions, dermatitis, burns, sunburn, torpid sores, decubitus X-ray incident sores, slow healing) requiring topical acute dressing (e.g., first-degree or higher degree burns, dermabrasions, postoperative or traumatic injury, amputations, skin grafts); chronic (e.g., limb ulcers, pressure sores or pressure ulcers, varicose ulcers, necrotic ulcers, traumatic ulcers, diabetic ulcers, neoplastic ulcers, necrosis); lesions characterized by the absence of healthy tissue surrounding the wound on which to apply adhesive surfaces, and infected and uninfected cancerous lesions, but can also be used on clean wounds or with minimal necrosis or infection during the early stages of healing, specifically in the stages of formation of granulation and epithelialization tissue. Lesions treated with the compressive dressing 1 are, by way of non-limiting examples: lesions with significant extension and depth; lesions presenting edges of various kinds (linear wounds, wounds with jagged and/or with lacerated-bruised edges); surgical lesions; contaminated lesions (“street” wounds, infected wounds, wounds with foreign bodies, etc.); bleeding/granular lesions; oozing/weeping lesions (with scarce, medium or abundant material).

The compressive dressing 1 has numerous advantages over known dressings in the art, due to the non-adhesiveness to the skin, the permeability to gases and impermeability to liquids, the ease of application and removal and the application of a uniform compression, homogeneous on the body part that it is wrapped around, reflecting an improvement of general clinical conditions as well as patient comfort that, even alone, he/she is able to correctly apply and remove the compressive dressing 1.

The fact that the dressing 1 allows achievement of a uniform and homogeneous compression is reflected in many advantages. Such a compression allows the maintenance of a good contact of the dressing with the wound bed and a good lymphatic drainage, thus limiting oedema, i.e. the accumulation of fluid in the extravascular tissue due to an alteration of the permeability of capillary walls and the pressure gradients present between the blood vessels and surrounding tissue. The external application of a uniform, homogeneous compression, favours, moreover, the blood circulation and the detachment of leukocytes from the endothelium impeding adhesion by preventing the formation of leukocyte plugs. It was also demonstrated that a uniform, homogeneous compression, is able to reduce the high levels of cytokines released as a result of the inflammatory process, thus promoting wound healing.

The dressing 1 is also produced with an anatomical conformation in different sizes to be used for dressing any part of the body of humans or animals with different physical structures. Moreover, the dressing 1 is radiolucent, non-magnetic, inert and thanks to these features it is not necessary to remove it to subject the patient to diagnostic tests involving the use of ionizing radiation or magnetic fields.

The advantages of the compressive dressing 1 as described and shown here lead to a reduction of the costs of hospital stays and at the same time a reduction of the costs incurred by the hospitals in the care of patients, as the healing of the patient is obtained with an overall reduced duration of treatment and therefore with a reduction in the number of days in hospital. The decrease in the average number of days of hospitalization of patients admitted is quantifiable in a range between 0.1% and 70%, up to 95%.

Furthermore, the decrease of the time taken for the application of the dressing and/or reduction in the number of human resources simultaneously engaged in the dressing phase of the patient, are quantifiable in a range between 20% and 80%, up to 95%.

It is therefore evident that a lower number of days in hospital for the healing of patients leads to an increase in the number of possible admissions with a consequent economic benefit to the public and private hospitals, to clinics and hospital facilities, for example, for the elderly.

The compressive dressing 1 can be applied directly onto the lesion entering into direct contact with the surface of the lesion (operating in this case as a primary dressing) and/or anchoring a primary dressing to the skin (operating in this case as a secondary dressing). When the compressive dressing 1 is used as a secondary dressing, it may be combined/coupled with other primary dressings.

When the compressive dressing 1 is used as a primary dressing, the elastic inner layer 10 compressive dressing 1 can optionally be soaked with or internally contain within it compounds with therapeutic properties, such as antibacterial, antifungal, anti-mould, anti-static. These compounds with therapeutic properties are commonly known to the industry.

Thanks to the excellent adhesion and to the greater convenience in positioning the compressive dressing 1 on the body part to be dressed, sac-like collections can be avoided of any therapeutically effective compounds applied to the skin and/or on the elastic inner layer 10 of the pressure dressing 1.

A further embodiment of the present invention relates to a production process, preferably but not necessarily, of the compressive dressing 1 with anatomical conformation, the method comprising:

i) providing a piece of a multilayer structure having at least a first and a second edge region 40 and 50 generally opposite, and at least a third and fourth edge region 41 and 51 generally opposite,

ii) implementing at least one intermediate slit in the piece by removal of a respective excess portion of the piece 102, 103, in such a way that at least two flaps 111, 112, 113 are defined in the piece, generally side by side and separated by said slit, wherein said slit, or each slit is created by making at least two intermediate cutting lines 201/202a, 202b/203 in the piece, which extend from the fourth edge region 51 toward the third edge region 41;

iii) approximating said at least two flaps 111 and 112, 112 and 113 to each another and binding them to each other along the two cutting lines 201/202a and 202b/203 of the respective slit, in the absence of substantial overlap between the flaps themselves, or rather, with the two cutting lines essentially remaining facing each other.

In one particular embodiment, the multilayer piece includes at least two intermediate slits, so that in the piece itself at least three flaps 111, 112, 113 are defined, generally side by side, the two outer flaps 111, 113 being bound to the central flap 112 as explained above, or rather, in the absence of substantial overlap.

Each slit—and therefore the respective excess portion 102, 103 removed from the piece—preferably has at least an approximately triangular profile.

In a further embodiment, the cutting lines 201/202a, 202b/203 can generally converge up to a vertex 120, 121 of the slit spaced from the third edge region 41 (as shown in FIG. 4).

One particularly advantageous embodiment of the compressive dressing is completed by applying relative closing means 60 along the first edge 40 and the second edge 50 of the piece, generally opposite to each other; in one possible embodiment, for this purpose a closure is employed of the type generally known as “hook and loop”, i.e. including a first strip 42 of “hairy” fabric or with loops along the first edge 40 and a second strip 52 of fabric with hooks or the like along the second edge 50, adapted to engage with the first strip 42.

Materials and Methods

The Elastic Inner Layer and Flexible Outer Layer.

The elastic inner layer 10 and the flexible outer layer 30 may be made from natural, synthetic and/or artificial materials, used singly or in combination.

The materials used for the realization of such elastic inner layers 10 and flexible outer layers 30 are preferably selected from: silk, cotton, cupro, lycra (spandex, elastam or elastan), elastane, micro modal, polyurethane foam, polyester (e.g. Axpet®), polyether, polypropylene, polyamide, silicone, nylon, polylactone, non-woven fabric, viscose, polyvinyl chloride, polyamide imide, polyamidoamine, polyethersulfone, polyvinyl alcohol, polyacrylonitrile, polyethylene oxide, polystyrene, polyvinylidene, polyvinylpyrrolidone, polyethylene.

The said materials, in the case wherein they do not inherently possess hydrophilic and absorbent properties necessary for the realization of the elastic inner layer 10, may be subjected to finishing treatments which give them the said features, among which we can mention for example plasma treatment.

The elasticity of the above materials can be obtained (starting from a non-elastic material), at an increased or decreased level (starting from an elastic material) by application of special construction techniques, i.e. weaving. By way of example cotton may have elasticity up to 80% of stretching as a function of the framework in which it is woven.

The Intermediate Elastic Layer

The intermediate elastic layer 20 can be made from natural, synthetic and/or artificial materials used singly or in combination.

The materials used are preferably selected from: polyurethane, polytetrafluoroethylene (e.g. Goretex®).

Such materials, as a function of the finishing process to which they are subjected, may acquire or modify the properties of water vapour permeability and impermeability to liquids, which are maintained even when these materials are stretched/elongated.

Compounds with Therapeutic Activity Associated to The Compressive Dressing

Compounds with therapeutic activity can be associated to the compressive dressing and preferably associated to the elastic inner layer 10 in contact with the skin and can be selected from natural and/or synthetic compounds, used singly or in combination.

Examples of compounds with therapeutic activity associated to the compressive dressing 1 are: polylactic acid, polycaprolactone, fibroin, chitin, cellulose, chitosan, gelatine, collagen of human, equine or other origin, hydrocolloid, hydrogel, Crabyon®.

Closing Formations, Materials and Methods of Application

The closing formations 60 are preferably made with Velcro straps.

The Velcro straps used are preferably made from natural, synthetic and/or artificial materials, preferably polypropylene and polyamide.

Examples of usable materials for the realization of Velcro straps are: PA6,6, PA6,6/PVC+Fe, PA12, lycra, polypropylene, polyester, polyethylene, vinyl.

The Velcro straps may be elastic or non-elastic.

The commercially available Velcro straps used in the present invention are: Klettolastic® from Gottlieb Binder GmbH & Co. KG or Velcro straps by Velcro® Hook 088, Loop 001, Vel-loop, PSI Hook, PSI Loop, Vel-loc 085, Super-Vel-Loc, Super-Vel-Loc quatrefoil, Velour (3165, 400, 3894, 3969, 3200), Ultra-mate (623, 720, 722, 736, 751, 759, 766, 805, 820, 830, 835, 839 848, 855, 866, 811), DCS#20, DCS#22, DCS#24, DCS#36, DCS#36NW, MVA8-E, ONE-WRAP.

The closing formations 60 can be applied with a degree of inclination from 0° to 90° or preferably from 0° to 45° with respect to the edge regions. For example with reference to FIG. 6, the closing formations 60 are applied with an orientation of 0° with respect to the edge regions 40 and 50 (i.e. they are parallel to the respective edge regions). In other embodiments the closing formations 60 can be applied in a perpendicular direction to the respective edge regions, or rather with an inclination of 90° relative to the respective edge regions.

The closing formations 60 may be applied in a single, double or multiple strips, for the entire length of the edges 40, 50, 41 and 51 of the multilayer structure or fractions of their lengths up to 0.1 mm.

In the case wherein the closing formations 60 are applied along the edge regions 40, 50, 41, 51 for fractions of their length, the closing formations 60 preferably have a length within the range 0.1 to 500 mm, ideally 5-50 mm.

In the case of application of double or multiple strips of the closing formations 60, the distance between a strip and an adjacent strip is less than or equal to 100 mm at the point of greatest proximity between the two strips.

The application of the closing formations, in particular in the form of Velcro straps, preferably takes place by heat sealing, electric welding, or ultrasonic, high frequency, or laser welding.

Using these techniques in place of the traditional stitching avoids puncturing the fabric and compromising the impermeability of the dressing.

Bonding Tapes; Materials and Methods of Application

The bonding tapes 82 to be used are made of natural, synthetic or artificial materials, used singly or in combination, elasticized or non-elasticized.

Examples of advantageously usable materials for the realization of the bonding tapes are: cotton, linen, silk, cupro, cellulose, lycra, polypropylene, polyurethane, polyurea, polyester, copolyester, polyether, polyamide (PA 6,6; PA6,6/PVC+Fe; PA 12), silicone, polylactone, non-woven fabric, polyurethane-based non-woven fabric, vinyl, viscose, polyvinyl chloride, polyamide imide, polyamidoamine, polyethersulfone, polyvinyl alcohol, polyacrylonitrile, polyethylene oxide, polystyrene, polyvinylidene, polyvinylpyrrolidone, polyethylene.

In general, the usable tapes have a weight greater than 10 g/m², preferably between 100 and 500 g/m².

The height of the tape may vary between 0.5 and 50 mm.

The thickness of the tape is contained within 0.01 mm and 10 mm, preferably between 0.1 and 1 mm. The bonding tapes 82 can be applied over the entire length of the bonding zones of the flaps or of fractions of it.

In general, the bonding tapes 82 are applied to the compressive dressing 1 alongside the outer surface 71 of the multilayer structure using, for example heat sealing techniques, or ultrasonic, high frequency or laser welding.

### Example 1

### The Multilayer Structure of the Compressive Dressing 1

In one particularly preferred embodiment, the multilayer structure of the compressive dressing 1 comprises:
An elastic inner layer 10 made from absorbent hydrophilic cotton, treated to prevent the release of particles, non-adhesive on the skin; this layer is made of a material purchased from the company Frizza S.p.A, trade name COTOMED (compact anti-torsion Mako Cotton S/Z, preshrunk product of superior quality, composition 100% cotton, weight 85-90 g/m²±5%);
An intermediate elastic layer 20 in polyurethane, permeable to gases and impermeable to liquids; this layer is made of a material purchased from the company Frizza S.p.A, trade name TEXIT (100% polyurethane composition, weight 40-60 g/m²±5%);
A flexible outer layer 30 in polyester; this layer is made of a material purchased from the company Frizza S.p.A, trade name Polymed (composition 100% micro-polyester, mesh 14 cm, ribs 15 cm, weight 140 g/m²±5%, dimensional stability in height and length −4/+2%, elongation at dynamometer ±15% (Zwick −3.6 kg).

The process of production of the multilayer structure of the compressive dressing 1 comprises, in a preferred embodiment, two phases:

i) the coupling of the flexible outer layer 30 in polyester with the intermediate elastic layer 20 in polyurethane thus obtaining a bilayer structure;

ii) the coupling of the bilayer structure with the elastic inner layer 10 in absorbent cotton, where this coupling provides the placing of contact of the elastic inner layer 10 in absorbent cotton wool with the intermediate layer 20 of the bilayer structure, thus obtaining a three-layer structure.

The coupling of the flexible outer layer 30 with the intermediate layer 20 and then the bilayer structure with the inner layer 10 is preferably carried out by heat sealing by depositing of micro dots of adhesive (preferably polyurethane glue); micro dots of glue are applied both in step i) and in step ii) on the intermediate layer 20. Subsequently the application of temperature and pressure on the bilayer structure or on the three-layer structure allows the adhesion between the layers (temperature approximately 100° C. at a flow rate of 10-15 m/min), the entire operation being carried out using specific equipment for HOT MELT lamination technology.

At the end of the coupling process by heat sealing, the multilayer structure is stored for a period of 24-48 hours during which the crosslinking is carried out.

The total weight of the multilayer structure is generally in the range of 50-800 g/m², preferably in the range 200-350 g/m². The total weight of the dressing is preferably greater than 10 g/m².

The overall thickness of the dressing 1 is generally in the range from 0.1 to 10 mm.

The elasticity of the dressing is preferably, but not necessarily, bidirectional.

For the evaluation of the elasticity, the procedure that is followed involves the use of a machine for traction testing (model Insight 1 kN; Manufacturer: MTS, Minnesota, USA), equipped with a 10N load cell and pneumatic grip loader. The speed of the test is 2 mm/min. The dressing samples were cut into sizes equal to 30 mm total length and 5 mm in width, the thickness of the sample is equal to 0.5 mm. Once inserted inside the pneumatic grip, the initial length (l₀) of the sample before the test is equal to 16 mm. Five samples of fabric were tested in the longitudinal direction and 5 samples of fabric in the transverse direction.

The elasticity is measured by considering parameters such as the initial length of the dressing (l₀) and the final length (l_{f}) after stretching. The elasticity was calculated by applying equation (eq. 1):

Δl%=[(l_{f}−l₀)/l₀]*100% (eq. 1).

The dressing 1 preferably has an elasticity, in the direction l of Δl % (transverse direction) up to 80%, preferably from 20% to 60%, and in the direction of the height h (longitudinal direction) up to 20%, preferably from 5% to 20%.

The dressing 1 can, however, have an inverse elasticity Δl % compared to the previous case, which is from 5% to 20% in the direction of the length l and from 20% to 80% in the direction of the height h.

In the case of the use of fabrics with weft and warp for the production of the dressing 1, the fabric is worked in such a way as to match the weft with the transverse direction of the dressing 1 and the warp with the longitudinal direction.

### Example 2

### Production of an Anatomically-Shaped Compressive Dressing for a Calf

For the production of an anatomically-shaped compressive dressing for a calf, it is necessary to create an “anatomical” cut of the multilayer structure.

With reference to FIG. 4, in a piece of the multilayer structure made according to example 1 and provided with at least four perimeter edges 40, 50, 41 and 51, two intermediate slits are created by removal of a respective excess portion of the piece 102, 103, in such a way that three flaps 111, 112, 113 are defined in the piece generally side by side and separated by said slits. These slits are made by carrying out at least two intermediate cutting lines 201/202a, 202b/203 in the piece that extend from the third edge 41 of the piece toward the fourth edge 51 of the piece opposite the third edge, the two cutting lines 201/202a, 202b/203 generally converging up to a vertex 120, 121 of the slit spaced from the third edge 41. Each slit—or rather the respective excess portion of 102, 103 of the piece that is removed—preferably has an approximately triangular profile.

Subsequently the three flaps 111 and 112, 112 and 113 are approximated to one another and joined together (e.g. by ultrasonic welding) along the two cutting lines 201/202a and 202b/203 of the respective slit in the absence of substantial overlap between the flaps themselves, or rather with the two cutting lines that essentially remain facing each other.

Subsequently, the bonding zones of the flaps are covered with bonding tapes 82 at the outer surface 71 of the multilayer structure, in turn coupled to the multilayer structure by welding, thus producing a multilayer structure with a three-dimensional profile adapted for conformation to a calf (FIG. 6).

The multilayer structure can be completed along the peripheral edges 40, 50, 41 and/or 51 by hemming or edging with an inelastic or elastic coating textile, which can be adhesive or non-adhesive, with or without trimming of the excess tissue. The edging can be produced in such a way as to embrace both a part of the inner surface 70 and a part of the outer surface 71 of the dressing 1. During the hemming operation the provision for the application of a tape (possibly elastic) is also possible on the only outer surface 71 of the dressing 1.

### Example 3

### Application of the Closing Formations in the Form of Velcro Straps

The Velcro straps used are preferably micro-Velcro straps GS03/306, and M5 GS03/66 from the Company M5 Biadesivi Ltd.

The micro-Velcro straps GS03/306, and GS03/66 are applied at the edges 40 and 50 of the multilayer-structure.

With reference to FIG. 1, a first strip 42 of micro-Velcro GS03/66 (loop) is applied (e.g. by ultrasonic welding) on the surface 70 of the multilayer structure, or rather at the elastic inner layer 10; a second strip 52 of micro-Velcro GS03/306 (hook) is applied (for example by ultrasonic welding) on the surface 71 of the multilayer structure, or rather at the flexible outer layer 30.

Alternatively, the Velcro straps GS03/66 and GS03/306 can be applied in a reversed manner with respect to what is described above.

### Example 4

### Resistance Evaporation Test RET

The compressive dressing 1 produced according to the examples 1 to 3 possesses evaporative resistance values in the range 0.1 to 100 m²·Pa/W/W (RET), preferably less than 10 m²·Pa/W (according to standard UNI EN 31092:1996).

The RET values were determined in the temperature range 35-45° C. and a relative humidity (RH) of 10-100% on an area of the dressing of a size in the range 50-900 cm².

The compressive dressing 1 produced according to the examples 1 to 3 generally has gas permeability values in the range 100-100,000 g/m² in 24 h, preferably 100-1,000 g/m² in 24 h.

The determination of the evaporative resistance (RET) to permeability is conducted according to UNI EN 31092:1996. The determination of RET is conducted using the methodology called Sweating Guarded Hot Plate (better known as Skin Model) of the Institute of Hoenstein.

This methodology requires that the compressive dressing 1 is placed on a plate heated to a temperature of 35° C. This pot is then saturated with water, which simulates the exudate of the wound, but is isolated from the compressive dressing 1 by means of a membrane, for example in Cellophane, which prevents the dressing from getting wet. The result is determined by the “resistance to evaporative heat loss” (abbreviated to RET−m²*Pa/W) and is expressed by the power required to maintain the plate at a constant temperature.

The determination is performed by placing the compressive dressing 1 with the surface 70 intended to be in contact with the skin, i.e. with the elastic inner side 10, against the heated plate.

The test conditions are: temperature 35±0.5° C., relative humidity 40±3%.

The compressive dressing 1 was introduced into a test chamber (the measurements were carried out in triplicate) leaving the dressing inside the test chamber for a time of about 20 minutes.

RET values in the range from 0.1 to 100 m²*Pa/W were also obtained at a temperature of 26° C. with contact with the heated plate for 15 minutes on a sample which had been previously maintained at 120° C. for 1 h or 135° C. for 45 minutes. The above conditions (exposure of the dressing to 120° C. for 1 h or 135° C. for 45 minutes) simulate one of the sterilization process to which the dressing 1 must undergo for its use.

The speed of transmission of water vapour (breathability) of the compressive dressing 1 was also evaluated according to the “inverted cup” methodology (standard UNI 4818-26:1992) to give a range of variation of this parameter equal to 100-100,000 g/m2 in 1 h. The inverted cup method evaluates, in a humid environment, the passage of grams of water vapour through a circular portion (diameter equal to about 7 cm) of the dressing 1, placed between two polytetrafluoroethylene (PTFE) membranes, all located above a layer of silica gel grains and below a layer of water maintained at 26° C. The test time is 1 h.

The transmission speed of the water vapour has also been evaluated in a dry air-conditioned room (according to standard UNI 4818-26:1992) and the variation range of this parameter is equal to 100-100,000 g/m² in 24 h.

To perform this test, cylindrical-shaped aluminium containers are used with a screw cap, where the lid has a hole of surface area of 1000 mm² (diameter 36 mm) to which the tissue sample is applied.

After pouring 25 ml of water into the container, the cylinder is placed in the inverted position so that the fabric sample is in contact with the water surface. Once the cylinder is weighed, it is placed in a desiccator for 24 hours. After 24 h the container is extracted from the desiccator, which is again weighed. The difference in weight defines the amount of water that has passed, in the form of steam, through the hole of the container, or rather through the fabric sample to be tested.

The index which allows measurement of the breathability of a fabric is the “speed of transmission of water vapour”, and is expressed in grams per square metre in 24 hours.

### Example 5

### Water Leakage Test

The dressing was subjected to a water leakage test, with increasing hydrostatic pressure.

The compressive dressing 1 presents values of resistance to the passage of water (according to standard UNI EN 20811:1993) greater than 1 cm H₂O, preferably greater than 300 cm H₂O, still preferably in the range 10-5000 cm H₂O, in conditions of temperature of 35-45° C. and relative humidity (RH) equal to 10-100% on a dressing area within a range equal to 50-400 cm².

The water leakage test (according to standard UNI EN 20811:1993) was conducted using a waterproof meter (FX 3000 HYDROTESTER III sold by the company TEXTEST AG) with increasing hydrostatic pressure, temperature of 20±2° C. and relative humidity of 65±4%.

The water leakage test was conducted by evaluating the permeability in the inside to outside direction of the dressing, i.e. by evaluating the permeability of the dressing from the elastic inner layer 10 in cotton towards the flexible outer layer 30 in polyester.

The dressing samples were housed inside a test chamber.

The water pressure was applied under the test chamber with an increase of pressure set at 60±3 cm H₂O/min corresponding to the most critical test condition and more closely resembling the situation of use of the dressing. The surface area of the dressing tested is equal to 100±1 cm². The tests were conducted five times.

In general, the method foresees the end of the test by the penetration of the third drop of water through the fabric.

### Example 6

### Absorbance Test

The compressive dressing 1 produced according to the examples 1 to 3 has absorbance values in the range of 0.1 l/m² to 5 l/m², preferably 0.5 l/m² to 2 l/m².

For the evaluation of the absorption capacity (and therefore of the hydrophilicity), the procedure followed involves the use of a balance Orma EB200 with an accuracy of ±0.1 mg. The test parameters are: temperature: 23±2° C.; humidity: 55±4% RH, test surface area: 9±0.1 cm².

Three fabric samples were used, three tests were performed for each sample.

Each sample was weighed in the “dry” condition, and then immersed in distilled water (water density considered equal to 1 kg/dm³) for a time equal to 5 seconds, was extracted and immediately weighed in the “wet” condition. At this point the difference between the average values of “wet” weight minus the “dry” weight was determined.

The absorbent capacity was then determined and found to be: 0.877±0.006 l/m².

Naturally, while the principle of the invention remains the same, the structural details and the embodiments may widely vary with respect to what has been described and illustrated merely by way of example, without departing 1 from the object of the present invention as specified in the following claims.

1. Compressive dressing applicable in a single winding to a body part, the dressing having an inner surface and an outer surface with substantially the same area, the dressing including:
a multilayer structure comprising
i) an elastic inner layer defining said internal surface and provided for contact with the skin, the elastic inner layer being hydrophilic, non-adhesive to the skin and of absorbent material,
ii) an elastic intermediate layer permeable to gases and impermeable to liquids, and
iii) a flexible outer layer defining said outer surface, wherein the multilayer structure has at least a first edge region and a second edge region generally opposite one another, and
closing formations mutually coupling at the first edge region and the second edge region to close the dressing as a single winding to the body part.

2. Compressive dressing according to claim 1, wherein the dressing has an elasticity of less than 80% in the transverse direction, preferably between 20% and 60%.

3. Compressive dressing if according to claim 1, wherein the dressing has an elasticity of less than 80% in the longitudinal direction, preferably between 20% and 60%.

4. Compressive dressing according to claim 1, wherein the distance between the first and second edge regions and/or between the closing formations and/or the overall elasticity of the multilayer structure is selected in such a way that the dressing exerts a compression of 1-60 mm Hg on the body part to be wrapped by the dressing.

5. Compressive dressing according to claim 1, wherein the dressing has an evaporation resistance within the range 0.1 to 100 m²Pa/W, preferably less than 10 m²Pa/W (UNI EN31092: 1996).

6. Compressive dressing according to claim 1, wherein the dressing has a resistance to the passage of water not less than 1 cm H₂O, preferably within the range 10-5,000 cm H₂O (UNI EN 20811:1993).

7. Compressive dressing according to claim 1, wherein the dressing has a permeability within the range 100-100,000 g/m² in 24 h (UNI EN 4818-26:1992).

8. Compressive dressing according to claim 1, wherein the dressing has closing formations mutually coupling at least at a third and a fourth edge region generally opposite one another of the multilayer structure, particularly to enable coupling between them of a plurality of dressings.

9. Compressive dressing according to claim 1, wherein the closing formations are in the form of Velcro.

10. Compressive dressing according to claim 1, wherein the dressing has an anatomical conformation, that is has a substantially three-dimensional profile configured as a function of the body part which the dressing is arranged for.

11. Compressive dressing according to claim 1, wherein the inner elastic layer contains at least one therapeutically effective compound for use in the treatment of a skin lesion.

12. Production process of a compressive dressing with an anatomical configuration, according to claim 1, the process including:
i) providing a piece of a multilayer structure having at least four edge regions, wherein a first edge region is generally opposite to a second edge region, and a third edge region is generally opposite to a fourth edge region;
ii) providing at least an intermediate slit in the piece by removing a corresponding excess portion of the piece, in such a way that at least two flaps are created in the piece generally facing one another and separated by said slit, wherein said at least one slit is created by making at least two intermediate cutting lines in the piece that extend from the fourth edge region of the piece to the third edge region of the piece,
iii) approximating said at least two flaps to one another and binding them together along the two cutting lines of the respective slit in the absence of a substantial overlap between the flaps or with the two cutting lines substantially facing one another, obtaining a compressive dressing with an anatomical conformation.

13. Process according to claim 12, wherein operation ii) includes making at least two intermediate slits in the multilayer piece to get at least three flaps generally facing one another.

14. Process according to claim 12, wherein the removed excess portions of the piece or the relative slit have at least an approximately triangular profile.

15. Process according to claim 12, wherein the two cutting lines generally converge up to a vertex spaced from the third edge region.

16. Process according to claim 12, further comprising the operation of:
iv) applying closing formations mutually coupling along the first edge region and the second edge region of the piece.
